(19) United States
(12) Patent Application Publication
(10) Pub. No.: US 2011/0129801 A1
(43) Pub. Date: Jun. 2, 2011

(54) COMPOSITIONS AND METHODS TO PREVENT AND TREAT DRY SOCKET POST-OPERATIVELY AFTER TOOTH EXTRACTION SURGERY

(76) Inventor: Shikha Pramanik Barman, Bedford, MA (US)

(21) Appl. No.: 12/626,866

(22) Filed: Nov. 27, 2009

(51) Int. Cl. A61C 5/04; A61K 31/716; A61K 9/10; A61Q 11/00

(52) U.S. Cl. 433/215; 514/54; 424/484

(57) ABSTRACT

The invention describes and claims compositions and methods for their use in the prevention and treatment of alveolar osteitis (dry socket) after tooth extraction surgery.

### FIELD OF THE INVENTION

This invention describes compositions and methods to prevent the occurrence of alveolar osteitis, also known as, “dry socket” from occurring, after the extraction of teeth, particularly impacted molars. These methods of prevention are based on the use of carefully designed biocompatible tissue-engineering matrices that absorb and retain the fibrin blood clot at the site of the extraction wound to provide an appropriate extra-cellular matrix for progenitor stem cells to attach, proliferate and differentiate into regenerated tissue. Also, described in this invention are methods to treat and ameliorate dry socket, after it has been clinically presented. These treatment modalities are based on drug-eluting matrices that control pain, eliminate infection on-site, lower inflammation and encourage subsequent healing of the tissues.

### BACKGROUND OF THE INVENTION

### Incidence of Alveolar Osteitis

Dry socket is the most common complication following a tooth extraction, with a peak incidence in the 40-45 year-old age group. The formation of Alveolar Osteitis (dry socket), also known as alveolitis sicca dolorosa, is the most common post-operative complication from extraction of third molars. Other synonymous terms are localized osteitis, postoperative alveolitis, alveolalgia, septic socket, necrotic socket, localized osteomyelitis and fibrinolytic alveolitis. The incidence of Alveolar Osteitis has been reported as 3-4% following routine dental extractions and ranges from 1% to 45% after the removal of mandibular third molars, 1, 2, 3, 4, 5. The incidence of dry socket is higher in the mandible, occurring up to 10 times more often for mandibular third molars compared to maxillary molars.

### Clinical Presentation

Clinical presentation of Dry Socket is as follows: post-operative pain in and around the extraction site, which increases in severity at any time between 1 and 3 days after the extraction, accompanied by a partially or totally disintegrated blood clot within the alveolar socket, with or without halitosis. Occasionally, the patients also complain of a very unpleasant taste (Halitosis). Alveolar osteitis is physically characterized by an empty tooth socket with exposed bone surfaces surrounded by inflamed tissue. The denuded alveolar bone results in extreme pain, irradiating from the empty socket, normally to the ipsilateral ear, temporal region or the eye. This condition is caused by failure of the blood clot tissue network to form in the socket post-operatively. The duration of dry socket ranges from 5-10 days. The extraction socket is usually devoid of clot with exposed bone that may be filled with food debris with edema of surrounding gingival tissue. The condition is usually self-limiting in healthy individuals. In persons with suppressed immune systems, presentation of dry socket must be treated. Pathogenesis of alveolar osteitis appears to result from the conversion of plasminogen to plasmin resulting in fibrinolysis of the blood clot within the extraction socket.

### Causative Factors

Factors attributed to the disruption in the healing of the extraction wound include trans-operative complications, presence of local infection, bacterial contamination of the socket, experience of the surgeon, contraceptive use, smoking, alcohol intake and use of local anesthetics with vasoconstrictors. Persons with diabetes mellitus, hormonal imbalances, antibiotics-induced immunosuppression, chemotherapy-induced immunosuppression, AIDS-related immunosuppression and radiation therapy-induced immunosuppression are especially susceptible to problems in the healing of the extraction wounds. Other factors such as smoking, excessive trauma to the tissue site, degree of impaction of the third molar, inadequate irrigation during and after extraction, oral conceptive use, timing in the menstrual cycle, use of an anesthetic with a vasoconstrictor, use of corticosteroids preoperatively, extraction-associated surgical trauma, and experience of the oral surgeon all have been identified as probable causes. Additional risk factors include presence of pericorontitis, high pre- and post-operative bacterial counts. Gender appears to play a role. Dry socket appears in 4.1% of women as compared to 0.5% of men.

### Disease Etiology

An increased incidence of alveolar osteitis occurs in the presence of peri-coronitis, peri-apical infection, periodontitis, gingivitis and in patients with poor oral hygiene 6. Nitzan at al. 7 showed a possible significance of anaerobic organisms (which are also the predominant organisms in pericoronitis) in relation to the etiology of dry socket. Increased fibrinolytic activity and the activation of plasminogen to plasmin in the presence of tissue activators have been implicated as causative factors. This increased fibrinolytic activity is thought to affect the integrity of the blood clot post-extraction. In a normal post-extraction socket, thrombin and fibrinogen together farm a fibrin clot over which the epithelium migrates. Then, during granulation tissue formation, new blood vessels grow into the clot and clot degradation occurs through the activity of fibroblasts and fibrinolysis via plasmin before the start of osteoproliferation. High plasmin-like fibrinolytic activities were noted from cultures of the anaerobe Treponema denticola, which is also known to be a putative micro-organism in the development of periodontal disease. It has been proposed that Treponema denticola was known to multiply and lyse blood clots. T. denticola is an anaerobic bacteria that has been identified in periodontal disease and is known produce the fetid odor and foul taste characteristic of dry socket. Finally, T. denticola exhibits plasmin-like fibrinolytic activity, while other common oral bacteria do not demonstrate these innate activities. T. denticola is also a late colonizer of the mouth, which provides further evidence that this bacteria may have implications in the presentation of dry socket.

### Methods Utilized to Prevent and Treat Dry Socket

Approaches used to prevent dry socket from occurring have included peri-operative use of chlorhexidine gluconate wash as a mouth rinse to prevent bacterial build-up within the socket, packing the socket with a gel comprised of 25% metronidazole, placement of locally applied gauze impregnated with chlorotetracycline ointment and use of other dry socket pastes 8, 9.

The prevention of alveolar osteitis has been focused on systemic and topical antimicrobial therapies. Chlorhexidine, povidone iodine, 9-aminoacridine, metronidazole, tetracycline and clindamycin have been used in both systemic and localized regimens as preventive measures for dry socket, with varying degrees of success. Alveolar osteitis is commonly treated by packing a eugenol-incorporated gelatin sponge into the empty socket until it is filled in with tissue. The packing is changed frequently (every 2-3 days) until the post-operative symptoms subside. The underlying common theme in all of the approaches thus far, is site-specific delivery of a medication or a combination of medications such as antibiotics or antiseptics via oral rinses or pastes. The success of these modes of treatments is varied and not robust across the wide variety of patient types undergoing tooth extraction due to the many underlying reasons that cause the development of this medical condition. A generalized approach toward site-localized delivery of antibiotics in the tooth socket does help in the prevention and treatment of localized infections, but it does not assist in the re-organization of tissue leading to the ultimate healing of the wound in the socket. On the contrary, some adverse effects are observed such as the development of antibiotic-resistant bacterial infections, foreign body reactions and physical blocking of the wound healing space due to presence of medication-containing pastes, etc. Furthermore, frequent use of oral rinses post-operatively can “wash” out fibrin clots requisite for wound healing at the site. Additionally, strong antiseptic and antibiotic containing rinses and pastes can lead to destruction of the oral mucosa, leaving the tissue vulnerable to fungal infections of the mucosa.

RELEVANT PTO DOCUMENTS

| | Title | USPTO I.D. # | Filing Date | Abstract |
|---|---|---|---|---|
| 1 | Prevention and | 20090081312 | Mar. 26, 2008 | A tissue adhesive antimicrobial material |
| | Treatment of | | | that is placed into a tooth extraction site |
| | Alveolar | | | for the sustained release of silver for the |
| | Osteitis | | | prevention and treatment of alveolar |
| | | | | osteitis. The antimicrobial material is |
| | | | | placed into a tooth extraction site via |
| | | | | syringe, hand instrument or hand |
| | | | | delivery device. |
| 2 | Technique for | 5,972,366 | Sep. 17, 1996 | A surgical implant or external wound |
| | the prevention | | | dressing which functions as both a |
| | of alveolar | | | hemostat and a device to safely and |
| | osteitis | | | effectively deliver any of a number of |
| | | | | pharmaceuticals to targeted tissue at a |
| | | | | controlled rate is disclosed. The device |
| | | | | generally comprises a carrier in the form |
| | | | | of fibers, sutures, fabrics, cross-linked |
| | | | | solid foams or bandages, a |
| | | | | pharmaceutical in solid micoparticulate |
| | | | | form releasably bound to the carrier |
| | | | | fibers, and a lipid adjuvant which aids |
| | | | | the binding of the microparticles to the |
| | | | | fibers as well as their function in the |
| | | | | body. |
| 3 | Bioresorbable | 20050036955 | Aug. 13, 2003 | A moldable, bioresorbable, |
| | tooth | | | biocompatible, non-allergenic |
| | extraction | | | crosslinked collagen derivative dressing |
| | socket dressing | | | for the prevention of post extraction |
| | | | | alveolar osteitis (dry socket) pain is |
| | | | | disclosed along with methods for use of |
| | | | | the gel. The dressing is placed at the |
| | | | | time of surgery acting as a bone |
| | | | | covering obtundant and physiologic |
| | | | | scaffolding for the conduction of normal |
| | | | | alveolar bone healing sequence of |
| | | | | fibroblast ingrowth, blood vessel |
| | | | | formation, and reossification of the |
| | | | | extraction site defect. In one form, the |
| | | | | dressing is a flowable, moldable, |
| | | | | biocompatible, bioresorbable dressing |
| | | | | prepared by reacting (i) a collagen |
| | | | | derivative, such as gelatin or |
| | | | | atelocollagen, and (ii) a non-cytotoxic |
| | | | | crosslinking agent. |
| 4 | Drug releasing | 5,972,366 | Sep. 17, 1996 | A surgical implant or external wound |
| | surgical | | | dressing which functions as both a |
| | implant or | | | hemostat and a device to safely and |
| | dressing | | | effectively deliver any of a number of |
| | material | | | pharmaceuticals to targeted tissue at a |
| | | | | controlled rate is disclosed. The device |
| | | | | generally comprises a carrier in the form |
| | | | | of fibers, sutures, fabrics, cross-linked |
| | | | | solid foams or bandages, a |
| | | | | pharmaceutical in solid micoparticulate |
| | | | | form releasably bound to the carrier |
| | | | | fibers, and a lipid adjuvant which aids |
| | | | | the binding of the microparticles to the |
| | | | | fibers as well as their function in the |
| | | | | body. |
| 5 | Bone implant | 3,952,414 | Oct. 29, 1974 | There is disclosed a method for the |
| | | | | prevention of osteitis and for the |
| | | | | prevention of atrophy of alveolar bone, |
| | | | | which comprises embedding an implant |
| | | | | into a boney cavity such as a cystic cavity |
| | | | | or an alveolus after a tooth extraction. |
| | | | | The implant is a body of a tissue- |
| | | | | compatible material and has a smooth |
| | | | | unbroken exterior surface defining a |
| | | | | bulbous, gibbous shape which generally |
| | | | | follows the contour of the cavity. It is |
| | | | | important that the material of the |
| | | | | implant be inert to the body. The |
| | | | | implant can be employed in cavities |
| | | | | which are too large to permit the normal |
| | | | | primary and secondary healing |
| | | | | processes to fill the cavity with |
| | | | | trabecular bone tissue. |
| 6 | Pharmaceutical | 4,882,149 | Sep. 21, 1988 | Pharmaceutical depot preparation for |
| | Depots | | | implantation into base tissue comprising |
| | | | | natural bone mineral from which the |
| | | | | naturally associated fat and bone- |
| | | | | proteins have been removed whereby |
| | | | | said bone is sterile and non-allergenic, |
| | | | | said bone mineral having absorbed |
| | | | | thereon and/or adsorbed therein one or |
| | | | | more physiologically active substances. |
| | | | | The physiologically active substance is |
| | | | | advantageously an antibiotic or |
| | | | | taurolidine or tauraltam or a protein or |
| | | | | polypeptide assisting bone regeneration. |
| 7 | Compositions, | 20060089584 | Oct. 28, 2005 | Dental dressing assemblies are formed |
| | assemblies, and | | | from hydrophilic polymer sponge |
| | methods applied | | | structures, such as a densified chitosan |
| | during or after | | | biomaterial. |
| | a dental | | | |
| | procedure to | | | |
| | ameliorate fluid | | | |
| | loss and/or | | | |
| | promote healing, | | | |
| | using a | | | |
| | hydrophilic | | | |
| | polymer sponge | | | |
| | structure such | | | |
| | as chitosan | | | |

### SUMMARY OF THE INVENTION

The invention described herein, describes compositions and methods toward the prevention of dry socket. The method also describes treatment of dry socket.

The methods and compositions described in this document prevent dry socket post-operatively, by: (a) use of a polymer-based scaffold to encourage formation of an extracellular matrix (ECM) for the progenitor cells to attach themselves to for cell proliferation and differentiation into new tissue, (b) prevention of fibrinolysis of existing fibrin clots, and (c) prevention of the development of infections at the site. This can be achieved by a combinatorial approach of tissue engineering (requisite for wound healing) and site-localized drug delivery of fibrinolytic inhibitors (requisite for inhibition of fibrinolysis) and antibiotics (requisite for infection control) which will synergistically prevent development of the “dry socket” syndrome. For cases where dry socket has already presented, treatment can be achieved by application of a drug-loaded medical dressing that also encourages wound healing.

Hallmark in the process of wound healing is the initiation of the healing response caused by the monocytes and macrophages. Fibroblasts and vascular endothelial cells in the implant site proliferate and begin to form granulation tissue. Depending upon the extent of the injury, granulation tissue may be seen as early as 3-5 days following implantation of a biomaterial. The wound healing response is generally dependent upon on the extent or degree of injury or defect created by the surgical procedure. Wound healing by primary union is the healing of clean, surgical incisions in which the wound edges have been approximated by surgical sutures. Wound healing by secondary union occurs when there is a large tissue defect that must be filled or there is extensive loss of tissue or cells. In the case of tooth extraction, wound healing by secondary union must occur by formation of granulation tissue. The repair of implant sites can involve two distinct processes: (1) regeneration, which is replacement of injured tissue by proliferative cells; and (2) persistence of the tissue framework or matrix. Initial formation of this early granulation tissue can be guided if the tooth extraction socket maintains an environment that would lead to maintenance and persistence of the progenitor cells, critical in the early stages in the wound healing process.

To achieve the criteria mentioned above, the clinical need exists for a sterile, biocompatible sponge-like polymeric matrix that “absorbs” the wound-associated blood cells and cellular and proteinaceous exudates, is non-irritating to gingival tissue, “acts as a scaffold” to encourage and promote the attachment of cell-adhesion proteins, prevents fibrinolysis and which bioabsorbs as new tissue grows in the socket.

1. Inclusion of 1-3 B Glucan in the Matrix

The “sponge-like” polymeric matrix would have incorporated 1-3 Beta D-Glucan. 1-3 Beta D-Glucan is a natural biopolymer that is known to stimulate the monocyte-macrophage system in humans. Beta-Glucans, originated from the outer cell wall of fungi are shown to have immune stimulatory activity, especially to enhance wound healing. In the wound healing process, the migration and proliferation of fibroblasts are essential. In cellular in-vitro, 1-3 B-D Glucan has been shown to enhance the proliferation of fibroblasts. Derivatives of 1-3 B D Glucan, such as aminated 1-3 B D-Glucan can be used for this purpose.

2. Inclusion of Polymers that Promote Cell Attachment

Polymeric compositions can be envisioned that are amenable to cell attachment such as polyvinyl alcohol of various molecular weights, or hyaluronates of various molecular weights, or chitin, chitosan, cellulose and derivatives, thereof. Also included in the polymeric matrix is collagen or collagen derivatives, which plays a critical role in the formation of microstructure of new regenerated tissue. Polymers that contain the RGD sequence and peptidoglycans can be used in the formation of the polymeric matrix.

3. Inclusion of Hemostatic and Fibrinolytic Components

The matrix can have components that have a hemostatic effect, encouraging the formation of blood clots in the extraction socket, thus providing the first step in wound healing. Certain disease conditions such as diabetes prevent proper wound healing, thus leading to a high percentage of alveolar osteitis in diabetics. Thus, inclusion of anti-fibrinolytics in the matrix may assist in the formation of a stable extracellular matrix requisite for new tissue generation.

What is claimed in this invention, are compositions and methods to prevent the occurrence of, as well as treat, alveolar osteitis (dry socket).

### DETAILED DESCRIPTION OF THE INVENTION

The medical dressing, or polymeric matrix, described in this invention, is designed to prevent dry socket by: (a) absorbing the wound fluid and retaining the blood clot at the site, (b) facilitating the attachment of progenitor stem cells to the matrix in order to encourage the process of tissue regeneration and wound healing, (c) preventing fibrinolysis of the fibrin clot, (d) preventing infection at the wound site during the wound healing process and (e) delivering an anesthetic locally, to manage pain associated with the tooth extraction. Alternatively, the medical dressing can be designed to treat dry socket by: (a) site-specific delivery of an antibiotic, (b) “covering” the exposed root surface of the dry socket with a biocompatible packing, its bioabsorbability modulated to the time required for wound healing.

The invention consists of a sterile drug-loaded medical dressing that can be placed post-operatively into the wound socket at the site or physical space created by the extraction of a tooth. The dressing will be placed into the socket space by a specially designed sterile, plastic applicator, which is designed to optimally “extrude” the pre-loaded sterile, dressing into the wound socket. The dressing can be used prophylactically, to prevent the formation of alveolar osteitis, or it can be used as a drug delivery device to site-selectively deliver medication to treat an infection, or to provide a sterile medicated dressing as a packing to treat dry socket, as the wound heals.

The applicator can be made out of medical plastic, such as HDPE or polypropylene, suitable for sterilization by gamma irradiation, steam sterilization or ethylene oxide sterilization, as appropriate. The applicator will be angled at an angle between 120-150 degrees, such that the clinician can easily place the dressing in the socket without handling the dressing.

In one embodiment of the invention, the “dressing” as defined above, consists of a dry, loosely constructed and porous “sponge” that is placed into the tooth socket by an applicator. The sponge will preferably have interconnected porosity of 10-200 microns, as in an open cell foam, where the free mobility of cellular components is possible. The sponge will be pliable with the tissue in the socket, and will expand to fit the socket space. This will secure the sponge “in place” after placement using an applicator. The sponge can be placed into the wound socket as a “dry sponge” that will rapidly absorb the fluid components of the extraction socket. Preferably, the sponge will replace the gauze that is typically placed at the site of the wound immediately after surgery. The sponge can be pre-fabricated as a hydrated sponge as well, to enable ease of placement and to minimize discomfort. After placement, the sponge absorbs the blood present at the site and retains the clot at the site.

The porous sponge presents a biological scaffold to which the progenitor stem cells at the site can attach and proliferate, thus providing a favorable environment for enhanced tissue regeneration and wound healing. The sponge will be of the “open-cell” type, which is defined as pores that are interconnected and not discrete. This would be necessary for cell attachment, cell communication and tissue regeneration, leading to organized wound healing. The pores of the sponge will be between 10-200 microns in mean diameter. The sponge will be constructed of biomaterials that support cell attachment and cell proliferation. Such materials can be selected from, but not limited to 1-3 B D-Glucan and derivatives, poly(lactide)-co-poly(glycolide)s, poly(ethylene oxide)-g-poly(lactide), poly(amine)-g-poly(lactide)s, poly(peptide)-co-poly(lactide), poly(caprolactone)-g-poly(lactide), and copolymers and combinations, thereof. Signaling molecules such as peptidoglycans can be incorporated. Other biomaterials that can be utilized for the construction of the “sponge” are polymers that promote cell attachment, such as B-Glucans, chitosan, hyaluronic acid, chrondroitin sulfate, polyamino acids and combinations thereof.

In another embodiment of the invention, the “dressing” may be applied as a solid, biocompatible extruded hydrogel containing quick-dissolving pore-forming constituents such as sodium chloride, sodium bicarbonate or other salts, thereof. The hydrogel may be thermosensitive, as in the poly(ethylene oxide)-co-poly(propylene oxide)-co-poly(ethylene oxide) polymers which are fluid at 4° C. and highly viscous gels at 37° C. The hydrogel may have chemically crosslinkable moieties such as acrylates and methacrylates, which can be crosslinked in the presence of a redox or photoinitiators. The hydrogel may be biodegradable and water-soluble, as well as crosslinkable. Such materials can be selected from, but not limited to polymers with a poly(ethylene oxide) backbone, chain extended by biodegradable ester linkages such as lactates and glycolates and end-capped with acrylates.

In another embodiment of the invention, the dressing may be a physical blend of several polymers to impart desired characteristics such as cell adhesion, sustained drug release, biocompatibility, biodegradability, pH adjustment, pliability, water absorption, equilibrium swelling and other physico-chemical attributes deemed necessary to achieve a dressing that would be non-irritating to the wound, prevent formation of the dry socket, promote wound healing and prevent local infections. For example, polymers such as cellulosics and polyesters (PLG, etc.) are amenable to cellular adhesion. Drugs such as chlorhexidine hydrochloride can be incorporated into the polymers to achieve sustained delivery of the medication to the site. Different salt forms of the drug may be used to slow down release from the dressing, altering the rate of dissolution to affect the release.

The polymers used to construct the dressing will be biodegradable, designed to bioabsorb by hydrolytic or enzymatic activity, within 3-7 days in the mouth.

The dressing may be comprised of synthetic biodegradable polymers to avoid having to remove the controlled release drug delivery system after use. There are numerous materials available for this purpose and having the characteristic of being able to break down or disintegrate over a prolonged period of time when positioned within the target tissue. As function of the chemistry of the biodegradable material the mechanism of the degradation process can be hydrolytic or enzymatic in nature, or both. The degradation preferably occurs either at the surface (heterogeneous or surface erosion) or uniformly throughout the drug delivery system depot (homogeneous or bulk erosion). Typically, to form biodegradable polymers, labile bonds are introduced in the polymer. Those labile bonds may be in the polymer backbone, so that cleavage creates low-molecular weight, water-soluble polymer fragments. The unstable bonds could also be part of a pendant side chain where the labile bond attaches an often hydrophobic side group to a water-soluble polymer. Furthermore, the unstable bonds could be part of a cross-linked network and upon cleavage in the cross-links producing soluble fragments.

Suitable materials to form the dressing are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof.

Suitable biodegradable polymeric materials include, by way of illustration, the widely studied esters of poly(glycolic acid) and poly(lactic acid) and their copolymers where the degradation rate is controlled by the ratio of glycolic acid to lactic acid, as well as copolyoxalates, poly(caprolactone), poly(lactide-co-caprolactone), poly(esteramides), polyorthoesters, polyanhydrides, polyacrylic acid, poly(lactide-co-glycolide) (PLG), poly(trimethylene carbonate), poly(glycolide), poly(ester-amides), poly(amides), poly(dioxanone), poly(y-ethyl glutamate), poly(DTH iminocarbonate), poly(Bisphenol A iminocarbonate), poly(sebacic acid-hexadecanoic acid anhydride), copolymers of poly(ethylene oxide)-poly(lactide) and derivatives, combinations and copolymers, thereof. Other suitable biodegradable materials include collagens; gelatin and pre-gelatinized starch; hyaluronic acid; polysaccharides such as calcium alginate; proteins such as albumin and fibrin; and combinations thereof. Numerous other biodegradable polymeric materials are well known to those of skill in the art and therefore the aforementioned list is not intended to limit the invention in any manner.

Suitable biodegradable non-polymeric materials include, by way of illustration and not limitation, natural and synthetic materials such as Vitamin E analogs such as the esters d-.alpha.-tocopheryl acetate and d-.alpha.-tocopheryl succinate. Vitamin E esters such as d-.alpha.-tocopheryl acetate and d-.alpha.-tocopheryl succinate are particularly well suited for use as a biodegradable non-polymeric depot material. These esters are solids at body temperature but have relatively low melt points (28.degree. C. and 76.degree. C., respectively). Therefore, the drug delivery system can be easily manufactured by melting the Vitamin E ester at a low temperature and the therapeutic agent can be admixed into the melt. The melt is then readily sub-divided into dosage units and cooled until solidified. Use of Vitamin E esters as the depot materials also provides additional benefits since the esters can also serve to stabilize instable therapeutic agents, as well as function as permeation enhancers to increase tissue absorption of the therapeutic agent. Numerous other biodegradable non-polymeric materials that can be utilized for this application are well known to those skilled in the art and therefore the aforementioned list is not intended to limit the invention in any manner.

Other polymers appropriate for this application may be cellulose derived polymers such as ethylcellulose (EC), hydroxyethyl cellulose (HEC), hydroxypropylcellulose (HPC), hydroxypropylmethylcellulose (HPMC) and methylcellulose (MC), as well as functionalized celluloses such as calcium carboxymethylcellulose, carboxymethylcellulose esters and sodium carboxymethylcellulose; poly(vinyl acetate) and so forth; chlorinated poly(ethylene); cross-linked poly(vinylpyrrolidone); ethylene-propylene rubber; ethylene-vinyl ester copolymers such as ethylene-vinyl acetate copolymer, ethylene-vinyl hexanoate copolymer, ethylene-vinyl propionate copolymer, ethylene-vinyl butyrate copolymer, ethylene-vinyl pentanoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer and ethylene-vinyl benzoate copolymer; natural rubber; plasticized poly(amides); plasticized nylon; plasticized poly(ethylene terephthalate); plasticized poly(vinylchloride); poly(acrylate); poly(acrylonitrile); poly(alkylmethacrylates) such as poly(methylmethacrylate) and poly(butylmethacrylate); poly(amides); poly(butadiene); polycarbamates or polyureas such as polyurethane polymers; poly(carbonates); poly(dimethylsiloxanes); poly(esters); poly(ethylene); poly(halo-olefins); poly(isobutylene); poly(isoprene); poly(4,4′-isopropylidene diphenylene carbonate); poly(tetrafluoroethylene); poly(trifluorochloroethylene); poly(methacrylate); poly(olefins); poly(oxides); poly(vinyls); poly(vinylidene chloride); and poly(vinyl-olefins); silicone; silicone-carbonate copolymers; silicone rubbers, particular medical grade; vinyl chloride-acrylonitrile copolymers; vinylidene chloride-acrylonitrile copolymers; vinyl chloride diethyl fumarate copolymers; and vinylidene chloride-vinyl chloride copolymers. Numerous other non-biodegradable polymeric materials are well known to those of skill in the art and therefore the aforementioned list is not intended to limit the invention in any manner.

The dressing may also be comprised of natural biopolymers such as hyaluronic acid, cellulosics, chitosan, chitin, amylose, pullulan, starch, glycosaminoglycans (GAGs) and combinations and derivatives thereof. Other biopolymers appropriate for this application may be proteins such as silk, keratin, collagen, gelatin, fibrinogen, elastin, actin and myosin. Furthermore, polymers such as chrondroitin sulfate, keratan sulfate, dermatan sulfate, heparin sulfate, and heparin can be utilized to fabricate the dressing.

The dressing may also be comprised of synthetic biocompatible polymers such as poly(ethylene oxide-b-propylene oxide-b-ethylene oxide), poly(ethylene oxide), copolymers of poly(lactide)-poly(ethylene oxide), etc. The polymers can have chemically crosslinkable moieties such as acrylates, fumarates, etc.

In another embodiment of the invention, the dressing may be a blend of water-soluble and water-insoluble polymers, wherein the water-insoluble polymer is a biodegradable polymer. Examples of water-insoluble biodegradable polymers are poly(lactide-co-glycolide), poly(lactide), poly(glycolide), poly(trimethylene carbonate) and copolymers thereof. Other examples are poly(ester-amides), poly(caprolactone), poly(butyrolactone), poly(propiolactone), etc. For example, the water-insoluble polymer can be spun into fibers and mixed with the water-soluble gel-forming polymer, then extruded as a hydrogel, post-surgery into the blood-laden tooth socket. The water-soluble hydrogel would dissolve away within an hour or two, leaving a mesh in place. The mesh acts like an open-celled matrix, providing cell-support for the progenitor cells to attach and re-organize into tissue. Sustained release of an antimicrobial compound such as chlorhexidine can be achieved by incorporation into the water-insoluble polymer mesh. The drug release is controlled by the rate of biodegradation of the insoluble polymer. In another example, blood clotting factors such as thrombin and fibrinogen can be incorporated into the water-soluble gel portion of the formulation, to promote clot formation in the tooth socket. In another embodiment of the invention, the hydrogel may be incorporated with an anesthetic to provide sustained release of the drug into the socket. This may include lidocaine, benzocaine, novocaine, and salts thereof. The anesthetic may be incorporated in the form of an insoluble salt to enable slow sustained release into the tooth socket.

In another embodiment of the invention, the dressing can be comprised of a pre-formed open-pore matrix “sponge” that can absorb excess blood oozing from the site and promote clot formation by “holding” the clot in place via the matrix. The sponge would be comprised of biodegradable water-soluble polymers with minimal swelling in water. The biodegradation time of the sponge will range from 3-7 days in the oral cavity, designed to degrade into non-toxic water-soluble components. The rate of hydrolytic degradation of the sponge will be modulated to coincide with the rate of tissue formation, as the healing process occurs. The sponge may have mucoadhesive properties, adhering to the tissue as a hydrated film molded to the shape of the socket. The “sponge” as is described herein, can be incorporated with an anesthetic, or an anti-microbial agent or an anti-fibrinolytic agent. Additionally, the sponge can be hydrated prior to placement, in an aqueous solution containing thrombin to promote wound healing and tissue re-organization.

In one application of the invention, the pre-formed “sponge” can be hydrated in an aqueous “hydrating” solution that is cold, to provide a soothing effect to the extraction site. The hydrating solution can also contain menthol, or peppermint for added soothing effect. The hydrating solution may also be incorporated with medicaments known to provide relief to gingival and periodontal tissue, such as Eugenol, Balsam of Peru, etc. The hydrating solution may contain water-soluble polymers that are of low viscosity when cold, and a physically-crosslinked hydrogel when warmed up to physiological temperature (37° C.). By “physically crosslinked” it is meant that the crosslinks are not covalent in nature (or chemically crosslinked), but gelled by intermolecular interactions such as hydrogen bonding, etc. Water-soluble gel forming polymers can be poly(ethylene oxide)-poly(propylene oxide)-poly(ethylene oxide), xanthan gum, carreegenan, guar gum, etc. The hydrating solution may be comprised of water soluble, non-gelling polymers of synthetic or natural origin. Examples of such polymers are cellulosic derivatives, chitosan derivatives, starch derivatives, etc. The hydrating solution may also be comprised of water soluble polymers that “gel” in the presence of metal ions. An example of this is sodium alginate, which forms a crosslinked gel in the presence of calcium ions.

In one method of manufacturing, a batch of the water-insoluble biodegradable polymer is first extruded into very fine fibers of diameter not greater than 0.5 mm. The fine fibers are then homogenously suspended into an aqueous solution containing the water-soluble polymer, filled into angled, wide-mouth syringes and lyophilized. Post-lyophilization, the syringes are capped. The syringes can be sterilized by ethylene oxide. In another method, the biodegradable polymer can be extruded and ETO-sterilized, then mixed in a sterile aqueous solution containing the water soluble polymer.

In one embodiment of the invention, bioactive agents or “medicines” can be incorporated into the dressing.

Exemplary tissue and bone growth factors to facilitate tissue and/or bone growth include by way of example and not limitation, growth hormones such as transforming growth factor-.beta. There are also other factors or enzymes such as alkaline phosphatase that are involved in the facilitation of tissue and/or bone regeneration. Alkaline phosphatase has been shown to be a biochemical indicator of bone turnover. Osteoblasts, generally regarded as bone forming cells, arise from marrow stroma cells. They are found on the surfaces where bone is being formed. Their most obvious function is to synthesize osteoid and collagen and control its subsequent mineralization. Both cytoplasm and nucleus of osteoblasts contain the enzyme alkaline phosphatase, which can be used as a marker for osteoblast activity. Alkaline phosphatase is a calcium- and phosphate binding protein that is distributed for example in periodontal ligament and more prominent in regions close to the alveolar bone and markedly lower in gingival connective tissue. Drugs, like dexamethasone, promote the differentiation of osteoprogenitor cells into osteoblasts and therefore can be used in lieu of growth factors.

Drugs like phenyloin (dilantin) can also be delivered to facilitate gum tissue re-growth. Phenyloin is an antiepileptic drug and is related to the barbiturates in chemical structure. Typically, it is administered to treat seizures and epilepsy. One of its pharmaceutical side-effects (gingival hyperplasia) can be used to enhance the gingival tissue regeneration.

What is claimed is:

1. A composition comprising one or more biocompatible polymers forming a bioabsorbable network or matix, 1-3 Beta D Glucan to facilitate the recruitment of network monocytes and macrophages for fibroblast proliferation, one or more agents to promote cell attachment, one or more agents to facilitate clot formation, one or more growth factors, one or more agents to inhibit fibrinolysis, one or more antibiotics, and one or more anesthetics.

2. A method for preventing or treating alveolar osteitis following tooth extraction wherein the composition of claim 1 is delivered into the tooth socket as a network-containing viscous liquid or a gel paste, whereupon the gel or viscous liquid slowly diffuses into the surrounding tissue, leaving the network in the socket.

3. A method for preventing or treating alveolar osteitis following tooth extraction wherein the composition of claim 1 is delivered into the tooth socket as a network-containing cold viscous liquid, which sets to a gel-like consistency after it reaches the physiological temperature of the mouth.

4. A method for preventing or treating alveolar osteitis following tooth extraction wherein the composition of claim 1 is delivered into the tooth socket as a porous dry packing, which slowly hydrates as it absorbs fluids from the tooth socket.

5. A composition as in claim 1 further comprising a fibrous network that has a sponge or foam microstructure with openings or channels that are interconnected, with channel diameters with a size distribution between 2 and 200 microns.

6. A composition as in claim 1 further comprising a biocompatible polymer component that is a physical mixture of water-soluble and water-insoluble polymers.

7. A composition as in claim 1 further comprising a network that encourages cell attachment and support, inter-cellular communication and signaling, and cellular migration and proliferation.

8. A composition as in claim 1 further comprising polymers that are reactive with blood components in order to encourage the initial establishment and subsequent development of the fibrinogen network in the socket.

9. A composition as in claim 1 further comprising microspheres that encapsulate a drug to achieve modulated release of the drug.

10. A composition as in claim 1 further comprising polymers that render the network bioabsorbable, degrading by hydrolysis or proteolysis, whereupon the bioabsorbability of the network is designed to bioabsorb proportionally with the rate of tissue regeneration in the wound.

11. A composition as in claim 1 further comprising polymers that are bioadhesive to the oral tissue.

12. A method for delivery of a composition as in claim 1 wherein the composition, is administered into the socket via an angled applicator, whereupon the angle of the applicator is between approximately 120-150 degrees, optimized for ease of delivery.
